(19) United States
(12) Patent Application Publication
(10) Pub. No.: US 2013/0079627 A1
(43) Pub. Date: Mar. 28, 2013

(54) AUGMENTED REALITY ULTRASOUND SYSTEM AND IMAGE FORMING METHOD

(71) Applicant: Jun-kyo LEE, Yangju-si (KR)

(72) Inventor: Jun-kyo LEE, Yangju-si (KR)

(73) Assignee: SAMSUNG MEDISON CO., LTD.

(21) Appl. No.: 13/243,076

(22) Filed: Sep. 23, 2011

(51) Int. Cl. A61B 8/13

(52) U.S. Cl. 600/424; 600/445

(57) ABSTRACT

An augmented reality ultrasound system. The augmented reality ultrasound system includes: a probe for transmitting an ultrasound signal to an object and receiving the ultrasound signal reflected from the object; an image generating unit for generating an ultrasound image from the ultrasound signal transmitted from the probe; a photographing unit for photographing the object and the probe to obtain images thereof and recognizing information corresponding to movement of the probe by using the image of the photographed probe; an image modifying unit for modifying the ultrasound image transmitted from the image generating unit so as to reflect the movement of the probe by using the movement information of the probe transmitted from the photographing unit; and a display unit for displaying the ultrasound image transmitted from the image modifying unit.

### BACKGROUND OF THE INVENTION

1. Field of the Invention

The present invention relates to an ultrasound system and an ultrasound image forming method, and more particularly, to an augmented reality ultrasound system and an augmented reality ultrasound image forming method.

2. Description of the Related Art

In a general ultrasound diagnosis apparatus, ultrasound delivered through a probe contacting a patient is reflected from the patient, and then the general ultrasound diagnosis apparatus receives the ultrasound to form an ultrasound image, and thus a user may determine a state of a part contacting the probe and diagnose the state. The probe includes one or more transducers to send an ultrasound pulse. When the ultrasound pulse collides against an object having different densities, a portion of the ultrasound pulse is reflected from the object and another portion of the ultrasound pulse is detected as an echo by the probe. A depth of cellular tissue at which the echo is generated may be calculated by measuring a time at which the echo is detected by the probe.

An ultrasound image shows an internal state of a part contacting a probe and changes according to movement of the probe. However, a general ultrasound diagnosis apparatus simply provides an ultrasound image according to the above-described ultrasound transmission/reception principle without considering a parameter such as a position, an angle, or a distance of a probe.

Also, when a general ultrasound apparatus is used, it is difficult for a patient to exactly recognize a part being shown in an ultrasound image.

### SUMMARY OF THE INVENTION

The present invention provides an augmented reality ultrasound system and image forming method that may show changes in an ultrasound image according to movement of a probe.

The present invention also provides an augmented reality ultrasound system and image forming method that may display an augmented reality ultrasound image in which an ultrasound image and a patient's image are matched with each other.

According to an aspect of the present invention, there is provided an augmented reality ultrasound system including: a probe for transmitting an ultrasound signal to an object and receiving the ultrasound signal reflected from the object; an image generating unit for generating an ultrasound image from the ultrasound signal transmitted from the probe; a photographing unit for photographing the object and the probe to obtain images thereof and recognizing information corresponding to movement of the probe by using the image of the probe; an image modifying unit for modifying the ultrasound image transmitted from the image generating unit so as to reflect the movement of the probe by using the movement information of the probe transmitted from the photographing unit; and a display unit for displaying the ultrasound image transmitted from the image modifying unit.

According to another aspect of the present invention, there is provided an augmented reality ultrasound image forming method including: forming an ultrasound image of an object; photographing the object and a probe on the object to obtain images thereof and recognizing information corresponding to movement of the probe by using the image of the probe; modifying the ultrasound image of the object so as to reflect the movement of the probe according to the movement information of the probe; and displaying the modified ultrasound image.

### BRIEF DESCRIPTION OF THE DRAWINGS

The above and other features and advantages of the present invention will become more apparent by describing in detail exemplary embodiments thereof with reference to the attached drawings in which:

FIG. 1 is a block diagram of an augmented reality ultrasound system, according to an embodiment of the present invention;

FIGS. 2A through 2C are views showing ultrasound images each transmitted from a probe having a bar code, according to embodiments of the present invention;

FIGS. 3A and 3B are views each showing an image of a probe and an ultrasound image matched with each other, according to embodiments of the present invention;

FIG. 4 is a view showing an image of an object transmitted from a photographing unit composed with a modified ultrasound image transmitted from an image modifying unit, according to an embodiment of the present invention; and

FIG. 5 is a flowchart showing an augmented reality ultrasound image forming method, according to an embodiment of the present invention.

### DETAILED DESCRIPTION OF THE INVENTION

Now, exemplary embodiments according to the present invention will be described in detail with reference to the accompanying drawings.

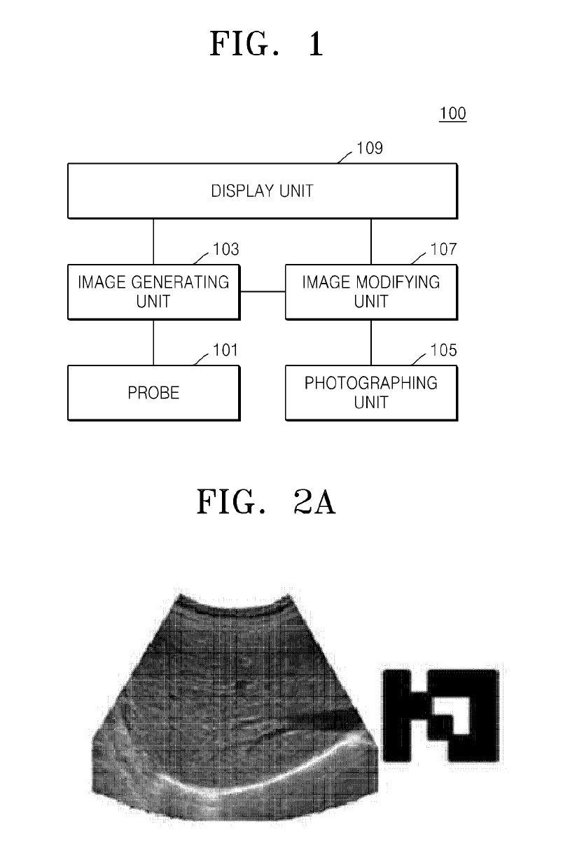

FIG. 1 is a block diagram of an augmented reality ultrasound system, according to an embodiment of the present invention.

Referring to FIG. 1, an augmented reality ultrasound system 100 includes a probe 101 for transmitting an ultrasound signal to an object and receiving the ultrasound signal reflected from the object, an image generating unit 103 for generating an ultrasound image from the ultrasound signal transmitted from the probe 101, a photographing unit 105 for photographing the object and recognizing movement information of the probe 101, an image modifying unit 107 for modifying the ultrasound image by using the movement information of the probe 101 transmitted from the photographing unit 105, and a display unit 109 for displaying the ultrasound image transmitted from the image modifying unit 107.

The probe 101 may send a movement information signal according to movement of the probe 101 with respect to a position at which a part of an object is contacted by the probe 101. For this, a bar code may be formed in the probe 101. However, any device capable of allowing movement of the probe 101 to be sensed may replace the bar code.

The image generating unit 103 generates an ultrasound image from an ultrasound signal transmitted from the probe 101. In the present embodiment, the ultrasound image may be a three-dimensional ultrasound image. For example, the image generating unit 103 may generate three-dimensional ultrasound data by using the ultrasound signal transmitted from the probe 101 and generate the three-dimensional ultrasound image based on the generated three-dimensional ultrasound data, but the present invention is not limited thereto. That is, the image generating unit 103 may generate a plurality of pieces of two-dimensional ultrasound data by using the ultrasound signal transmitted from the probe 101 and generate the three-dimensional ultrasound image based on the generated plurality of pieces of two-dimensional ultrasound data.

The photographing unit 105 may be a video camera that radiates visible light or infrared light onto an object. An image transmitted from the photographing unit 105 is a real time image or a still image of the object.

The photographing unit 105 recognizes and extracts movement information of the probe 101, that is, information regarding at least one selected from the group consisting of a position, an angle, and a distance of the probe 101, at the same time that an object is photographed, and transmits the information to the image modifying unit 107.

The image modifying unit 107 executes at least one modifying operation selected from the group consisting of rotation, upsizing, and downsizing of an ultrasound image according to movement information of the probe 101. When the ultrasound image is a three-dimensional ultrasound image, the image modifying unit 107 may modify the three-dimensional ultrasound image according to the movement information of the probe 101.

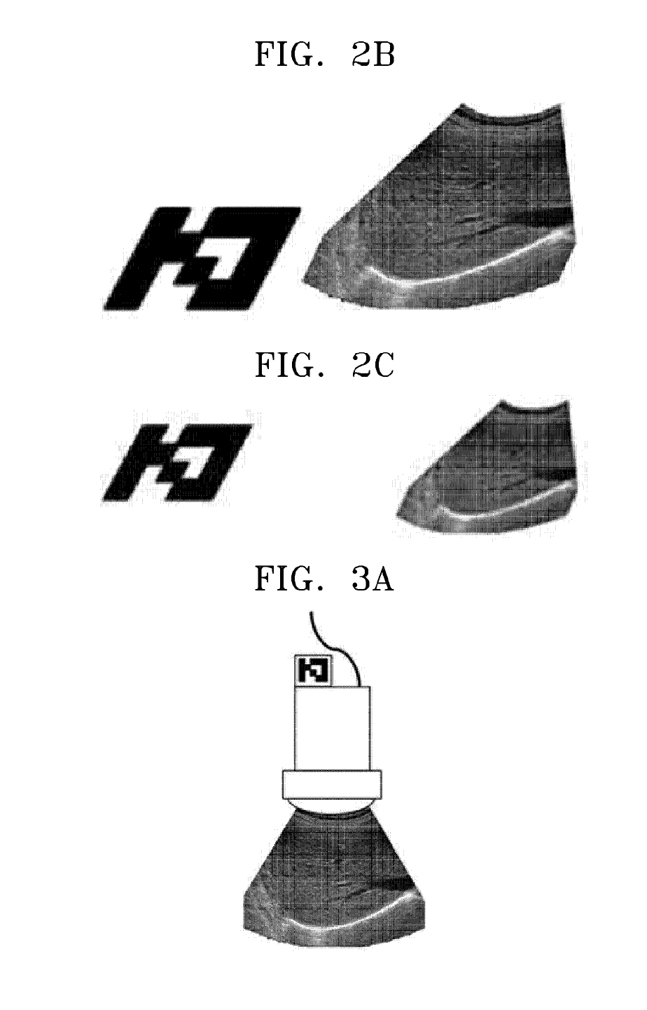

FIGS. 2A through 2C are views showing ultrasound images each transmitted from a probe having a bar code, according to embodiments of the present invention.

FIG. 2A is a view showing an ultrasound image when the bar code has not been modified. However, as the probe moves, the ultrasound image may be modified as illustrated in FIGS. 2B and 2C. Referring to FIG. 2B, the bar code is tilted to the right and right and left sides of the bar code are enlarged with respect to the bar code shown in FIG. 2A, and thus the ultrasound image is rotated at a predetermined angle to the right and is enlarged. In FIG. 2C, the bar code is downsized with respect to the bar code shown in FIG. 2B. Thus, the ultrasound image shown in FIG. 2C has the same tilt as that of FIG. 2B, but is downsized in all directions.

Thus, the augmented reality ultrasound system according to the current embodiment of the present invention includes a device capable of rapidly recognizing modification of a bar code according to movement of a probe and transmitting movement information, and thus the augmented reality ultrasound system may provide an augmented reality ultrasound image with a sense of reality by modifying an ultrasound image in real time according to movement of the probe.

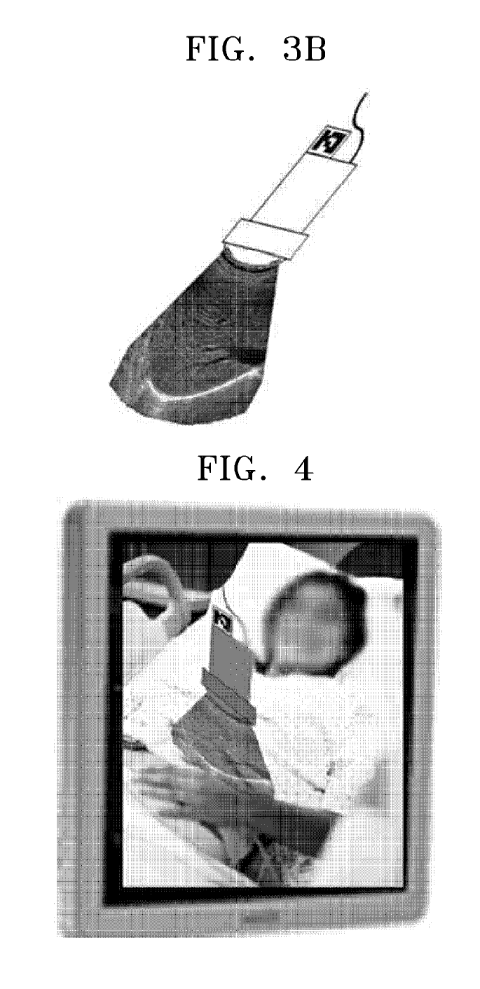

FIGS. 3A and 3B are views each showing an image of a probe and an ultrasound image matched with each other, according to embodiments of the present invention.

Referring to FIG. 3A, the ultrasound image modified according to movement of the probe, as illustrated in FIGS. 2A through 2C, is composed with the image of the probe so as to be displayed with a sense of reality in the display unit 109. In FIG. 3B, a bar code is rotated to the right according to movement of the probe and is downsized, compared to FIG. 3A. In conjunction with a signal obtained due to modification of the bar code, the image of the probe may be rotated to the right, may be downsized, and may be matched with the ultrasound image. In this regard, it is assumed that a modification operation, such as those illustrated in FIGS. 2A through 2C, has been already performed on the ultrasound image. From such an augmented reality image modification technology, a sense of reality of the ultrasound image may be further enhanced. In general, modification of the ultrasound image may be at least one selected from the group consisting of upsizing, downsizing, and rotation. However, the ultrasound image may be modified in various other ways, for example, composition with a piston image, composition with a bar code image, or composition with a diagnostic image.

The display unit 109 may display an image formed by composing an image of an object transmitted from the photographing unit 105 with an ultrasound image transmitted from the image modifying unit 107. Alternately, the display unit 109 may further include a supplementary display unit (not shown) for displaying only the ultrasound image, and may additionally compose the image transmitted from the photographing unit 105 with the ultrasound image from the image modifying unit 107 and display the composed image. That is, if the display unit 109 may display an ultrasound image modified according to movement of the probe 101 as described with respect to the augmented reality ultrasound system 100 according to the current embodiment of the present invention, the type and number of display units 109 are not limited.

FIG. 4 is a view showing an image of an object transmitted from the photographing unit 105 composed with a modified ultrasound image transmitted from the image modifying unit 107, according to an embodiment of the present invention.

Referring to FIG. 4, the modified ultrasound image is composed and matched with an abdomen on the image of the object, which is a patient. The patient watching the display unit 109 may intuitively understand the abdomen is being shown in the ultrasound image.

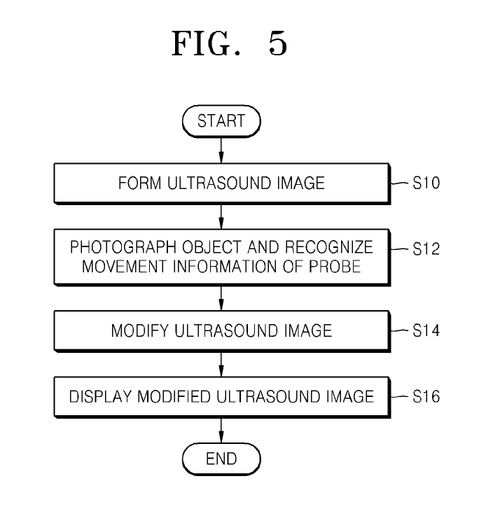

FIG. 5 is a flowchart showing an augmented reality ultrasound image forming method, according to an embodiment of the present invention.

First, an ultrasound image of an object is formed (S10). A probe for transmitting/receiving an ultrasound signal may be a device for recognizing information corresponding to, for example, movement of a bar code of the probe. The ultrasound image may be a three-dimensional ultrasound image, but the present invention is not limited thereto.

Second, the object is photographed, and the movement information of the probe with respect to the object is recognized (S12). In this regard, an image obtained by photographing the object may be a real time image or a still image obtained by radiating visible light or infrared light on the object. The movement information of the probe may be information regarding at least one selected from the group consisting of a position, an angle, and a distance of the probe.

Third, at least one modification operation selected from the group consisting of rotation, upsizing, and downsizing is performed on the ultrasound image of the object according to the movement information of the probe (S14). Finally, the modified ultrasound image is displayed (S16). Alternatively, although not shown in FIG. 5, the augmented reality ultrasound image forming method may further include composing of the image obtained by photographing the object with the modified ultrasound image and displaying of the composed image to display an augmented reality ultrasound image.

Accordingly, the augmented reality ultrasound system and the ultrasound image forming method according to the embodiments of the present invention may provide a live ultrasound image because an ultrasound image may be modified in real time according to movement of a probe. Furthermore, the augmented reality ultrasound system and the ultrasound image forming method according to the embodiments of the present invention may allow users to intuitively understand the ultrasound image by composing an image of a probe with a patient's image and displaying the composed image, and may provide a diagnosis result having high reliability.

According to the embodiments of the present invention, a realistic ultrasound image may be provided in real time by rotating, upsizing, and downsizing an ultrasound image according to movement of a probe.

Also, according to the embodiments of the present invention, a patient's image is matched with an ultrasound image so as to provide an augmented reality ultrasound image that may allow the patient to intuitively recognize a diagnosis result.

The augmented reality ultrasound image forming method according to an embodiment of the present invention may be implemented in a program command form that may be executed through various computer elements and may be recorded in a computer readable recording medium. The computer readable recording medium may include program commands, data files, data structures, etc., individually or in combination. The program command recorded in the computer readable recording medium may be a program command designed specifically for the present invention or may be a program command well-known to one of ordinary skill in the art. Examples of the computer readable recording medium include hard disks, floppy disks, magnetic media such as a magnetic tape, optical media such as a compact disk read-only memory (CD-ROM) or a digital versatile disk (DVD), magneto-optical media such as a floptical disk, and hardware devices such as read-only memory (ROM), random-access memory (RAM), or flash memory, formed specifically to store and execute program commands. Examples of the program command include machine codes made by a compiler and high-level language codes that may be executed by a computer by using an interpreter. The aforementioned hardware devices may include one or more software modules in order to execute operations of the present invention.

While the present invention has been particularly shown and described with reference to exemplary embodiments thereof, it will be understood by those of ordinary skill in the art that various changes in form and details may be made therein without departing from the spirit and scope of the present invention as defined by the following claims.

What is claimed is:

1. An augmented reality ultrasound system comprising:
a probe for transmitting an ultrasound signal to an object and receiving the ultrasound signal reflected from the object;
an image generating unit for generating an ultrasound image based on the ultrasound signal transmitted from the probe;
a photographing unit for photographing the object and the probe to obtain images thereof and recognizing information corresponding to movement of the probe by using the image of the probe;
an image modifying unit for modifying the ultrasound image transmitted from the image generating unit so as to reflect the movement of the probe by using the movement information of the probe transmitted from the photographing unit; and
a display unit for displaying the ultrasound image transmitted from the image modifying unit.

2. The augmented reality ultrasound system of claim 1, wherein the image modifying unit executes at least one selected from the group consisting of rotation, upsizing, and downsizing on the ultrasound image transmitted from the image generating unit.

3. The augmented reality ultrasound system of claim 1, wherein the image modifying unit composes and matches the modified ultrasound image with the image of the probe.

4. The augmented reality ultrasound system of claim 1, wherein the image modifying unit composes and matches the modified ultrasound image with the image of the object.

5. The augmented reality ultrasound system of claim 1, wherein the display unit composes the image of the object transmitted from the photographing unit with the image of the probe or composing the image of the object with the ultrasound image transmitted from the image modifying unit, and displays the composed image.

6. The augmented reality ultrasound system of claim 1, wherein the photographing unit transmits a real time image or a still image.

7. The augmented reality ultrasound system of claim 1, wherein the photographing unit radiates visible light or infrared light onto the object.

8. The augmented reality ultrasound system of claim 1, wherein the probe comprises a bar code, and the photographing unit photographs the bar code of the probe to obtain an image thereof and recognizes the movement information of the probe by using the image of the bar code of the probe.

9. The augmented reality ultrasound system of claim 1, wherein the movement information of the probe includes information regarding at least one selected from the group consisting of a position, an angle, and a distance of the probe.

10. The augmented reality ultrasound system of claim 1, wherein the ultrasound image is a three-dimensional ultrasound image.

11. An augmented reality ultrasound image forming method comprising:
forming an ultrasound image of an object;
photographing the object and a probe on the object to obtain images thereof and recognizing information corresponding to movement of the probe by using the image of the probe;
modifying the ultrasound image of the object so as to reflect the movement of the probe according to the movement information of the probe; and
displaying the modified ultrasound image.

12. The augmented reality ultrasound image forming method of claim 11, further comprising:
composing the image obtained by photographing the object with the image obtained by photographing the probe or composing the image obtained by photographing the object with the modified ultrasound image, and displaying the composed image.

13. The augmented reality ultrasound image forming method of claim 12, wherein the image obtained by photographing the object is a real time image or a still image.

14. The augmented reality ultrasound image forming method of claim 11, wherein, in the photographing of the object and in the recognizing of the movement information of the probe, the object is photographed by radiating visible light or infrared light onto the object.

15. The augmented reality ultrasound image forming method of claim 11, wherein the probe comprises a bar code, and the recognizing of the movement information of the probe comprises photographing the bar code of the probe to obtain an image thereof and recognizing the movement information of the probe by using the image of the bar code of the probe.

16. The augmented reality ultrasound image forming method of claim 11, wherein the movement information of the probe comprises information regarding at least one selected from the group consisting of a position, an angle, and a distance of the probe.

17. The augmented reality ultrasound image forming method of claim 11, wherein, in the modifying of the ultrasound image of the object according to the movement information of the probe, at least one modification operation selected from the group consisting of rotation, upsizing, and downsizing is performed on the ultrasound image.

18. The augmented reality ultrasound image forming method of claim 11, wherein the modifying of the ultrasound image of the object according to the movement information of the probe comprises composing the modified ultrasound image with the image of the probe.

19. The augmented reality ultrasound image forming method of claim 11, wherein the ultrasound image comprises a three-dimensional ultrasound image.

20. A computer readable recording medium having embodied thereon a computer program for executing the method of claim 11.
